(19) United States
(12) Patent Application Publication
(10) Pub. No.: US 2007/0050027 A1
(43) Pub. Date: Mar. 1, 2007

(54) Leak-proof breast implant

(76) Inventors: Donald K. McGhan, Las Vegas, NV (US); Jim J. McGhan, Las Vegas, NV (US); C. Scott Eschbach, Las Vegas, NV (US)

Correspondence Address: MICHAEL G PETIT, 123 W PADRE ST., SUITE F, P O BOX 91929, SANTA BARBARA, CA, 93190, US

(21) Appl. No.: 11/508,078

(22) Filed: Aug. 21, 2006

Related U.S. Application Data
(60) Provisional application No. 60/711,255, filed on Aug. 24, 2005.

(51) Int. Cl. A61F 2/12

(52) U.S. Cl. 623/8

(57) ABSTRACT

A leak-proof implantable breast prosthesis and a method for making the prosthesis. The breast prosthesis comprises an elastically deformable body or core which is encased within a flexible biocompatible elastomeric shell. In one embodiment, the deformable body is attached, in segments, to the inner surface of the shell by adhesive means and has shape-memory. In another embodiment, the shell encases the core but is not attached to the core. When the outer shell of the prosthesis is subjected to a deforming force, the prosthesis deforms then returns to an original shape when the deforming force is removed. In the event that the shell is ruptured, the prosthesis cannot leak exogenous filler fluid into the surrounding tissue because the prosthesis is substantially void of exogenous filler fluid.

This application claims the benefit of U.S. Provisional Application No. 60/711,255, filed Aug. 24, 2005.

### BACKGROUND OF THE INVENTION

1. Field of the Invention

The present invention relates to an implantable prosthesis for augmenting or replacing soft tissue of the breast, and, more particularly, to a leak-proof breast prosthesis, and even more particularly to a method for making an implantable leak-proof breast prosthesis.

2. Prior Art

Breast implants currently in use for prosthetic purposes comprise an elastomeric shell, usually a cured silicone elastomeric envelope, containing a fluid or gel filler. The shell is sealed to contain the filler within the shell and prevent leakage of the gel or fluid filler into surrounding tissue. The two most commonly used filler materials are silicone gel and isotonic saline fluid. Silicone gel-filled prostheses have aesthetic advantages, and their implantation can contribute to a more desireable result than the implantation of saline-filled breast implants. Recently, consideration has been given to the potential for rupture of the shell of the prosthesis. If the shell is ruptured following implantation, the filler material may leak from within the confines of the shell and become exposed to the surrounding tissue.

If the shell of a saline-filled implant is ruptured and the filler leaks into the surrounding tissue, the saline is absorbed by the body, and the primary function of the prosthesis is compromised due to loss of shape and volume. If the shell of a silicone gel-filled implant is ruptured, the filler may or may not remain within the intended location of the implant, but the surrounding tissue may be directly exposed to the silicone gel. A perception by some that the extrusion of silicone gel from a ruptured prosthesis may pose a potential danger to the recipient (whether or not such a perception has any validity) has influenced some physicians and patients to retrench to the aesthetically inferior saline-filled prosthesis. Even intact silicone gel-filled implants demonstrate diffusion of liquid silicone materials across the shell membrane and cause exposure of surrounding tissue to components of the silicone gel. The perceptions of potential danger to the recipient extend to this phenomenon, as well. Accordingly, there is a continuing need for a breast prosthesis that provides an aesthetically satisfactory implant, that minimizes or eliminates the diffusion of filler material components out of the shell, and which cannot leak if ruptured.

### SUMMARY

The present invention is directed to an impantable, leak-proof breast prosthesis that substantially obviates one or more of the limitations of the implants disclosed in the related art. To achieve these and other advantages and in accordance with the purpose of the invention, as embodied and broadly described herein, the invention is an implant comprising a solid, space-filling, elastically deformable core affixed to and encased within a biocompatible, preferably elastomeric shell. In a preferred embodiment, the core is an elastically deformable body having elastic or “shape” memory comprised of a plurality of segments affixed to the inner surface of a flexible shell.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1 is a transverse cross-sectional view of a breast implant in accordance with a first preferred embodiment of the present invention wherein the core has a hollow cavity therein and is encased within a shell.

FIG. 2 is a transverse cross-sectional view of a breast implant in accordance with a second preferred embodiment of the present invention wherein the core has a hollow interior cavity and an opening in the base thereof.

FIG. 3 is a transverse cross-sectional view of a breast implant in accordance with a third preferred embodiment of the present invention wherein the core is a hollow, self-supporting breast-shaped body which may or may not include a hole in the base thereof.

FIG. 4 is a transverse cross-sectional view of a breast implant in accordance with a fourth preferred embodiment of the present invention wherein the core is a solid, self-supporting breast-shaped body.

FIG. 5 is a transverse cross-sectional view of a breast implant in accordance with a fifth preferred embodiment of the present invention wherein the core has an opening in the base thereof, as shown in FIG. 2, but wherein the outer shell encases both the outer surface of the core and the hollow interior cavity of the core.

FIG. 6 is a transverse cross-sectional view of an everted breast implant in accordance with a sixth preferred embodiment of the present invention wherein the core has an opening in the base thereof, as shown in FIG. 2, and wherein the core, which has an irregular topography, is first applied to the outer surface of the shell prior to everting the shell to form an implantable breast prosthesis.

FIG. 7 is a transverse cross-sectional view of a breast implant in accordance with FIG. 6 wherein after the core is affixed to the shell, the shell is everted to form an implantable breast prosthesis.

FIG. 8 is a perspective view of the everted breast implant of FIG. 6 illustrating the affixation of a segment of the core to the outer surface of the shell prior to everting the shell to form the breast implant.

### DESCRIPTION OF THE PREFERRED EMBODIMENTS

A breast has a shape which is more or less personal to an individual. When a breast is deformed, such as during a mammogram wherein the breast is compressed between parallel plates, and the compressive force is removed, the breast returns to it's original (i.e., predeformed) shape. This indicates that not only is breast tissue deformable, but that the breast has a shape memory. Accordingly, a breast prosthesis used for augmentation or reconstruction should, itself, preferably exhibit the same properties of elastic deformability and shape memory as exhibited by the breast.

Breast prostheses that are in current use comprise a fluid filler encased in a thin, flexible silicone envelope. Saline and silicone gel are preferred filler materials for such prior art breast implants. Both filler materials contain fluid components and unfortunately; both filler materials can release fluid from within the shell under the appropriate circumstances, such as mechanical trauma. An elastomeric foam body, either alone or encased within a biocompatible outer shell, has been proposed for forming a breast prosthesis. While such an implant would be leak-proof provided that the foam material does not contain a fluid component, a disadvantage of a foam implant is that the density of the foam body and the mechanical properties (elasticity, modulus, etc.) of the foam body are different from the density of breast tissue and the prosthesis does not provide an aesthetically desirable result when surgically implanted in the human body.

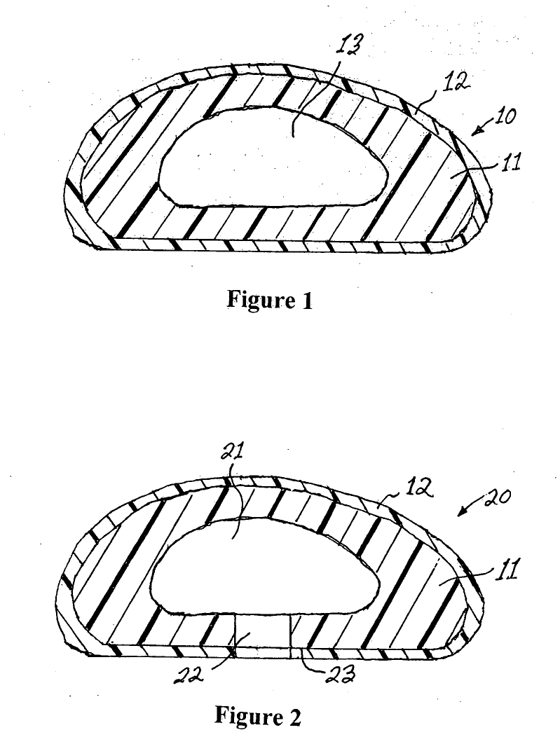

FIG. 1 is a transverse cross-sectional view of a novel breast implant 10 in accordance with a first preferred embodiment of the present invention wherein the core 11 has a hollow cavity 13 therein and is encased within a shell 12. The elastomeric core 11 has shape memory. The elastomer core 11 is elastically deformable and has a density approximating the density of breast tissue and a high cohesiveness. The elasticity of the elastomeric core 11 approximates that of the natural, human breast, including its breast tissue and overlying skin. In the first preferred embodiment 10, the elastomeric core 11 is encased within a thin flexible shell 12 comprising a biocompatible elastomer such as silicone.

FIG. 2 is a transverse cross-sectional view of a breast implant 20 in accordance with a second preferred embodiment of the present invention wherein the core 11 is encased in an elastomeric shell 12 and has a hollow interior cavity 21 and an opening 22 in the base 23 thereof. In FIG. 2, the opening 22 in the core 11 is shown open, providing a conduit between the cavity 21 and the external environment, but may be sealed by the shell 12. If the core 11 and/or shell 12 is made by dipping a breast-shaped mandrel in an elastomer in the manner well known in the art, the opening 22 provides means for removing the core and/or shell from the mandrel.

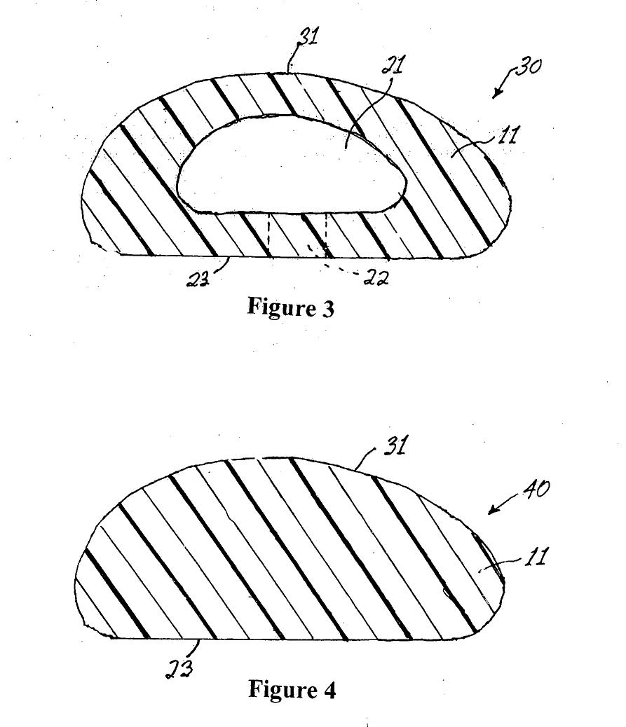

FIG. 3 is a transverse cross-sectional view of a breast implant 30 in accordance with a third preferred embodiment of the present invention wherein the core 11 is a hollow, self-supporting elastically deformable breast-shaped body which may or may not include a hole 22 (shown in phantom) in the base 23 thereof. Since the outer surface 31 of the core 11 will be in contact with tissue following implantation, the core 11 comprises a biocompatible elastomeric gel, most preferably a silicone gel.

FIG. 4 is a transverse cross-sectional view of a breast implant 40 in accordance with a fourth preferred embodiment of the present invention wherein the core 11 is a solid, self-supporting, elastically deformable breast-shaped body. The outer surface 31 of the core 11 in either of the embodiments 30 or 40 may be cured to a greater degree than the interior portion of the core such as, for example, briefly exposing the outer surface to heat, to reduce or prevent the migration of plasticizers from the core 11 through the outer surface of the prosthesis 40.

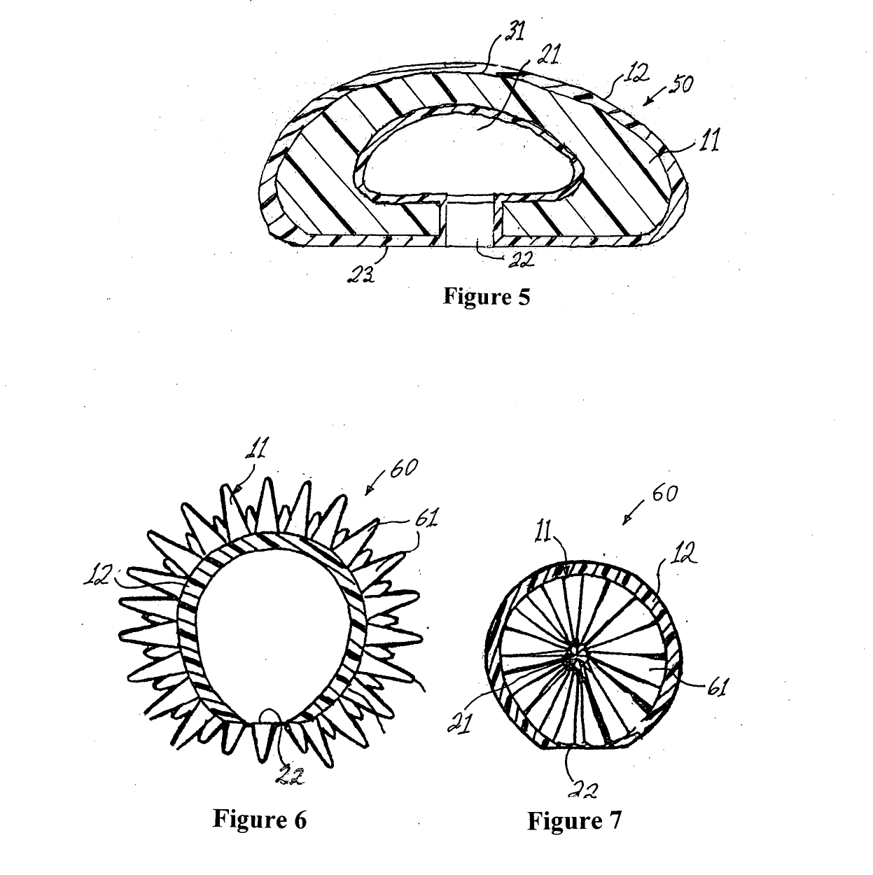

FIG. 5 is a transverse cross-sectional view of a breast implant 50 in accordance with a fifth preferred embodiment of the present invention wherein the core 11 has an opening 22 in the base thereof, as shown in FIG. 2, but wherein the biocompatible outer shell 12 encases both the outer surface 31 of the core 11 and the hollow interior cavity 21 of the core 11. Accordingly, if, following implantation, host tissue ingrowth occurs within the cavity 21, all surfaces of the prosthesis 50 contacting such invading host tissue are biocompatible.

It may be desirable to make a prosthesis as described above but wherein the core has an irregular topography projecting into the cavity. FIG. 6 is a transverse cross-sectional view of a breast implant 60 in accordance with a sixth preferred embodiment of the present invention wherein the prosthesis 60 is illustrated in an everted configuration. In embodiment 60, both the core 11 and the shell 12 have an opening 22 in the base thereof which enables the flexible elastomeric shell 12 to be everted after the core 11 is adhered to the surface of the shell 12. In the everted configuration of embodiment 60 illustrated in FIG. 6, the core 11 is elastically deformable and has an irregular topography comprising a plurality of protruberances 61 on an outer surface thereof. When the core 11 is affixed to the outer surface of the shell 11 as shown in FIG. 6, the implantable form of the breast prosthesis is formed by everting the configuration illustrated in FIG. 6 to provide the breast prosthesis illustrated in FIG. 7.

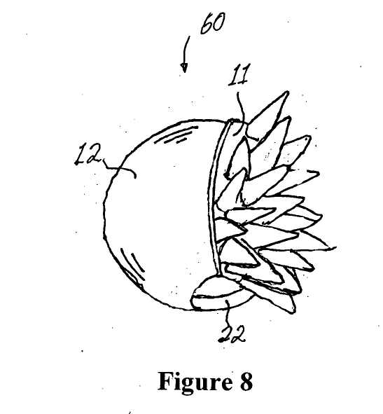

The embodiment of the breast prosthesis 60 shown in FIG. 7 is conveniently made by the process comprising the steps of: (a) forming a flat sheet of core material having the desired surface topography, then (b) cutting segments from the core sheet: (c) adhering the segments to the outer surface of the shell as illustrated in FIG. 8, until the outer surface of the shell is substantially covered with the core segments 11; then (d) after the core segments 11 have been adhered to the outer surface of the shell 12, the shell is everted to form an implantable breast prosthesis 60 as shown in FIG. 7. FIG. 7 is a transverse cross-sectional view of the breast implant 60 wherein after the core segments 11 are affixed to the shell, the shell is everted to form an implantable breast prosthesis 60. FIG. 8 is a perspective view of the everted breast implant 60 of FIG. 6 illustrating the affixation of a segment of the core sheet to the outer surface of the shell prior to everting the shell to form the breast implant.

The elastomeric core may be prepared from a variety of elastomeric materials, including thermoset polymers such as natural or synthetic rubbers or elastomers prepared from polyurethane, silicone, neoprene, isoprene, butadiene, chloroprene, isobutylene, EPDM, tetrafluoroethylene, hexafluoropropylene, and others. The elastomer core may also be prepared from thermoplastic elastomers, such as those prepared from polyurethanes, copolyesters, styrene copolymers, polyolefins, and others. The core may comprise a nonbiocompatible elastomer having the desired elastic deformability and other properties. In the latter embodiment, the core must be isolated from contact with tissue by encapsulation within at least one biocompatible barrier layer such as, for example, adhering a silicone coating on the outer surface thereof, or the nonbiocompatible core may be encased within a biocompatible silicone shell. The core preferably has unitary construction and may be modified in a variety of ways to provide the most desired characteristics. For example, the overall density and deformability of the core may be changed by removing plugs of material from the core. Alternatively, mechanical treatment of the core, such as crushing, abrading, or slitting may be employed to provide the desired characteristics to the core. As described above, the core may be made by first forming a flat core sheet having the desired thickness and topography from a biocompatible elastomeric composition in accordance with methods well known in the art, then cutting segments from the core sheet and adhering the segments to the surface of the shell until the surface of the shell is covered with core, then everting the shell to encase the core.

Elastically deformable silicone gel compositions having shape memory are disclosed, for example, in U.S. Pat. No. 6,743,843 to Fujisawa and U.S. Pat. No. 4,172,298 to Rechenberg. The latter patent discloses a silicone gel composition for use in a breast prosthesis for external use. It is expected that further compositions will be developed that provide a core having a suitable consistency and density.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What we claim is:

1. A breast implant comprising a solid, elastically deformable core enveloped in a flexible biocompatible outer shell.

2. A breast implant of claim 1, in which the solid, elastically deformable envelope is encased in a separate biocompatible shell.

3. A breast implant of claim 1, in which the flexible biocompatible shell is integral with the solid, elastically deformable core.

4. A breast implant of claim 3, in which the flexible biocompatible shell is formed by treating the surface of the solid, elastically deformable core.

5. A breast implant of claim 1, in which the solid, elastically deformable core contains less than 10% by weight of material capable of migration from the implant into surrounding breast tissue.

6. A breast implant of claim 5, in which the solid elastically deformable core contains less than 5% by weight of material capable of migration from the implant into surrounding breast tissue.

7. The breast implant of claim 1 wherein the elastomeric core has a cavity therewithin.

8. The breast implant of claim 2 wherein the elastomeric core has a cavity therewithin.

9. The breast implant of claim 4 wherein the elastomeric core has a cavity therewithin.

10. A method for making an implantable prosthesis having a solid core encased within a flexible biocompatible shell comprising the steps of:
(a) presenting a flexible elastomeric shell made from a biocompatible elastomer, said shell having a hole therein;
(b) presenting a flat sheet of a biocompatible, elastically deformable core material;
(c) cutting segments of core material from said flat sheet of core material; then
(d) adhering said segments of core material to an outer surface of said shell until said shell is substantially covered with said core material; then
(e) everting said shell through said hole in said shell such that said segments of said core material adhered to said shell are encased within said shell to form an implantable prosthesis.
